(19) United States
(12) Patent Application Publication
(10) Pub. No.: US 2017/0188808 A1
(43) Pub. Date: Jul. 6, 2017

(54) METHOD OF PRESCRIBING/MAKING EYEWEAR FOR AN INDIVIDUAL

(71) Applicant: Michael QUIGLEY, Montreal (CA)

(72) Inventor: Michael QUIGLEY, Montreal (CA)

(21) Appl. No.: 15/340,033

(22) Filed: Nov. 1, 2016

Related U.S. Application Data
(63) Continuation of application No. PCT/CA2015/050403, filed on May 7, 2015.
(60) Provisional application No. 61/989,670, filed on May 7, 2014.

(51) Int. Cl. A61B 3/00; A61B 3/117; B05D 5/06; G02C 7/10; B05D 1/18; A61B 3/103; A61B 3/10

(52) U.S. Cl. CPC A61B 3/0025; A61B 3/103; A61B 3/117; A61B 3/1005; G02C 7/10; B05D 1/18; B05D 5/06

(57) ABSTRACT

A method of prescribing eyewear for an individual. The method includes determining a relative retinal illuminance level for the individual and selecting, on a basis of the determined retinal illuminance level, a light transmission characteristic for eyewear lenses for the individual. At least one refractive parameter of the individual's eye is measured in order to determine the individual's retinal illuminance level.

### CROSS-REFERENCE TO RELATED APPLICATION

This application is a continuation of PCT/CA2015/050403 filed on May 7, 2015 that claims priority from U.S. Provisional Application Ser. No. 61/989,670 filed May 7, 2014, and which is hereby incorporated herein by reference.

### TECHNICAL FIELD

The present invention relates generally to the field of prescription eyewear and, more specifically, to a method of prescribing eyewear for an individual.

### BACKGROUND

Light, travelling through space in the form of waves at different wavelengths, some visible, some invisible to the human eye, can have damaging effects on the eye. For example, ultraviolet (UV) waves, found between x-rays (10 to 190 nm) and visible light (380 to 780 nm), are too short for the human eye to detect but have a great amount of radiant energy and can reach the eye directly or indirectly (e.g. sunlight reflected off the surface of water, sand, snow or other bright objects reflects light radiation to the eyes). Overexposure to UV radiation and/or to High Energy Visible (HEV) light (which is in the violet/blue band (380 to 530 nm)), can cause significant damage to the retina and may contribute to eye disease, including cataracts, macular edema and possibly age-related macular degeneration (ARMD).

Sunglasses are used by adults and children alike and offer sun protection to the skin around the eye, the ocular surface, and the lens, thereby decreasing the risk of skin cancer around the eye, damaged conjunctiva (pterygion and penguicula) and cataracts, respectively. Proof of retinal protection by sunglasses has to date not been established. There is no standardization of the properties or characteristics of sunglasses and they are usually chosen for comfort level. In general, the basic characteristics are tint darkness, tint color, polarization and photochromism. Darkness can vary from about 8% transmissivity for extreme dark sunglasses (e.g. mirror coated glasses for skiing) to 90% transmissivity for bright, indoor use. Tint color is commonly neutral gray, but can be brown or green. The choice of sunglasses and protective glasses is personal and often related to comfort level and vision with a mix of tint, color, polarization and photochromic properties that affect transmission properties.

ARMD, which is hypothesized to be the result of photochemical damage affecting the macula (central vision portion of the retina), is the leading cause of blindness in persons over the age of 50. A person with ARMD loses his or her central vision and the condition slowly worsens over time, the damage to the eye irreversible in most cases. In the general population, it has been determined that myopic eyes (wearing concave corrective lenses) have less age-related macular degeneration (ARMD) than hyperopic eyes (wearing convex corrective lenses) and this in a dose-response manner (see “Refractive errors and age-related macular degeneration: a systematic review and meta-analysis”, Pan et al., Ophthalmology, 120(10), 2058-2065, 2013). The cause of ARMD is unclear but is theorized, on the basis of laboratory studies, to be instigated by light damage to the retina. However, several major epidemiologic studies (e.g. The Beaver Dam Eye Study by Tomany et al (Arch Ophthalmol, 122(5), 750-757, 2004) and the POLA study by Delcourt et al (Arch Ophthalmol, 119(10), 1463-1468, 2001)) have declared that ARMD does not appear to be associated with sunlight exposure. Nevertheless, both of these studies have acknowledged that the wearing of sunglasses leads to a statistically significant protective effect on certain retinal abnormalities seen in ARMD.

Although well-designed, well-fitted sunglasses can block UV and HEV light, the shade or level of tint of the lenses might not necessarily be appropriate to provide optimum protection for the person wearing the sunglasses. Retinal brightness (or intensity of light at the retina, also referred to as retinal illuminance) varies from person to person; as such, the effect of an individual's respective retinal brightness on his/her chances of incurring retinal damage over time, as well as the associated level of ocular protection required to reduce this risk, may also vary from person to person.

Variable-tint lenses (photochromic lenses) are used in transition-style sunglasses and darken upon exposure to certain kinds of light, most commonly UV radiation. As the light source increases or diminishes in intensity, the tint of the lenses gradually darkens or lightens, respectively. In the case of transition-style glasses for indoor/outdoor use, when the light source is removed completely (i.e. by moving indoors), the lenses gradually return to a clear state. Unfortunately, photochromic lenses respond only to the level of UV radiation for adjusting their tint level; they do not and cannot adjust their tint level on a basis of the brightness sensitivity (retinal luminance) of the individual wearing the glasses. As such, the protection afforded by the variable-tint lens is limited, since it cannot adapt to each individual's respective required level of retinal protection.

In U.S. Pat. No. 7,204,591, issued Apr. 17, 2007 to Wertheim et al, there is disclosed a device using light emitting diodes directed at a patient as a light source for evaluating the appropriate color and density of filters or sunglasses for the patient. The device enables a practitioner to determine which filter or sunglass colors and densities provide the optimal vision for the patient, under various lighting conditions. The device allows a practitioner to prescribe a sunglass color/density that will provide the best vision for the patient, this prescription does not consider the retinal illuminance of the patient and might not be optimum for reducing the patient's particular risk of incurring retinal damage.

Accordingly, there exists a need to provide a method for prescribing and manufacturing eyewear that will protect an individual's ocular health on a basis of a retinal illuminance level for the individual.

### SUMMARY

It has been discovered that eyewear tint can be selected based on an individual's level of retinal illumination.

In some embodiments, a method of prescribing eyewear for an individual comprises determining a relative retinal illuminance level for the individual and selecting, on a basis of the determined retinal illuminance level, a light transmission characteristic for eyewear lenses for the individual.

In some embodiments, a method of prescribing eyewear for an individual comprises measuring at least one refractive parameter of the individual's eye, and using it (or several refractive eye parameters) to determine a minimum tint level for eyewear lenses for the individual.

In some embodiments, a computer-implemented method of calculating a light transmission characteristic for eyewear lenses for an individual comprises inputting measured refractive parameters of the eyes of the individual, calculating the individual's retinal illuminance level based on a model of the eye, and identifying a minimum tint level for the eyewear lenses.

In some embodiments, a method of manufacturing eyewear lenses for an individual comprises using the individual's relative retinal illuminance based on that individual's eye refractive parameter(s) to identify a minimum tint level for the lenses, producing a pair of lenses to fit an eyewear frame selected by the individual, and applying at least said minimum tint level to the lenses by a tinting process.

### BRIEF DESCRIPTION OF THE DRAWINGS

The invention will be better understood by way of the following detailed description of embodiments of the invention with reference to the appended drawings, in which:

FIG. 1 is a sectional side view illustrating the anatomy of the eye;

FIG. 2 is a schematic optical diagram of the eye;

FIG. 3 is a table of eye setting details and optical design software measurement results, in accordance with a non-limiting example of implementation of the present invention;

FIG. 4 illustrates a data set showing the correlation of eye refractive parameters to retinal illuminance, as output by an optical design software program, in accordance with a non-limiting example of implementation of the present invention;

FIG. 5 is a plot of retinal illumination as a function of axial length, in accordance with a non-limiting example of implementation of the present invention;

FIG. 6 is a plot of retinal illumination as a function of eyewear prescription dioptres, in accordance with a non-limiting example of implementation of the present invention; and

FIG. 7 is a table showing a tint transmission level as a function of measured axial length, in accordance with a non-limiting example of implementation of the present invention.

### DETAILED DESCRIPTION

The present invention is directed to a novel method for prescribing and manufacturing eyewear for an individual, such as to reduce the individual's risk of incurring retinal damage due to overexposure to UV radiation and/or HEV light (or any other potentially damaging wavelength).

Note that the eyewear may be any type of eyewear with lenses, including eyeglasses, sunglasses, protective goggles, contact lenses and intraocular lenses. The lenses may be made of plastic or glass, without departing from the scope of the present invention.

In the context of the present specification, “retinal illuminance” is defined as a measure of image “brightness” on an individual's retina. More specifically, “retinal illuminance”, also referred to herein as “photonic intensity”, is a measure of the number of photons of light (or light energy) falling on the macula per unit area, which may also be described as a measure of the amount of photonic energy hitting the macula.

In the context of the present specification, the term “tint” is defined as any of various lighter or darker shades of a color that attenuate light transmission (especially high energy light).

In the context of the present specification, the term “refractive parameter” (or “refractive variable”) is defined as a component of the eye that influences how light, or any other radiation, propagates through the eye. Specific examples of refractive parameters of an eye include the axial length of the eye, the corneal refractive power and the lens correction (e.g. spectacle correction, contact lens correction or intra-ocular lens correction), among other possibilities.

By calculating retinal illuminance (intensity of light or photonic energy, in rays or photons/mm²) in an average hyperopic eye (wearing a convex corrective lens) and an average myopic eye (wearing a concave corrective lens), it has been determined that retinal illuminance (or retinal illumination) is significantly greater in an average short axial length hyperopic eye than in an average long axial length myopic eye. As such, light intensity, or the total amount of photonic energy, at the retina is related not only to the intensity of the source but also correlates strongly with the respective refractive parameters (or variables) of the eye, such as axial length (eye-globe diameter), corrective lens power (e.g. spectacle power) and corneal power. Since hyperopic eyes are known to have more ARMD than myopic eyes, it follows that an individual's specific retinal illuminance (i.e. the total amount of photonic energy hitting the individual's retina) may affect that individual's risk of developing retinal damage and/or diseases, such as ARMD, over time.

Accordingly, specific to the present invention, there is provided a novel method for prescribing and manufacturing eyewear for an individual on a basis of a determined retinal illuminance level for the individual. Basically, in a broad embodiment of the invention, the individual's relative retinal illuminance level is computed based on the eye's refractive components (or a combination of these components including spectacle correction, corneal power, and axial length) and then used to prescribe a light transmission characteristic (such as, for example, a photon reduction level or a tint level) of eyewear lenses for the individual, such as to decrease the photonic intensity in the individual's eyes. In a non-limiting example of implementation, a minimum tint level for eyewear lenses for the individual is selected on a basis of at least one refractive parameter of the individual's eyes, as discussed in further detail below.

Advantageously, by determining an individual's retinal illuminance level, it is possible to identify a minimum degree of retinal macular or retinal protection (also referred to herein as a retinal protection factor (RPF) value, or a macular protection factor (MPF) value) required by that individual in order to reduce the individual's respective chances of incurring retinal damage due to exposure over time of the macula to high levels of photonic energy (e.g. from UV radiation and HEV light). This MPF value can then be transformed into a recommended minimum tint level for the lenses of eyewear worn by the individual, particularly eyewear worn outdoors or under conditions of exposure to natural light.

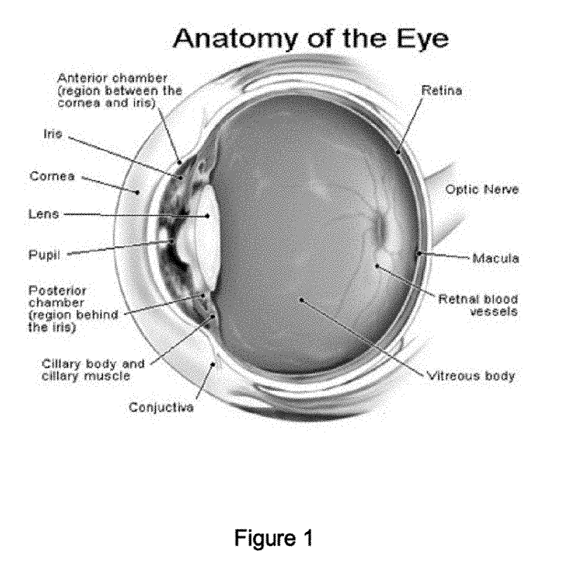

As seen in FIG. 1, the human eye is formed of a plurality of different layers and optical components, including the cornea, the iris, the pupil, the lens and the retina. A plurality of different refractive parameters of the eye combine to determine a total refractive error of the eye. Examples of such refractive parameters include the corneal power (refractive power of the cornea), the lens power (refractive power of the lens to focus light onto the retina), the anterior chamber depth (depth of the fluid-filled space between the iris and the cornea's innermost surface) and the axial length (distance between the anterior surface of the cornea and the center of the macula region of the retina).

In a specific non-limiting example of implementation of the present invention, the retinal illuminance (RI) level, or photonic intensity level, of an eye is calculated on a basis of the throughflux (T) and (linear) image magnification (IM) of the eye, as follows:

RI=T/IM² (energy/cm²)

where T and IM can be computed on the basis of at least one measured refractive parameter of the eye, as discussed below.

The pertinent refractive components, namely spectacle power, corneal power, and axial length can be arrived at using routinely available devices available in optometric, ophthalmic or optometric clinics. For example, the lensometer for spectacle measurement, the keratometer for corneal measurement, or an optical coherence biometer like the Carl Zeiss IOL Master. Less commonly today, ultrasound can be employed to measure the eye's axial length.

For illustration purposes, two simple model eyes (also referred to as simplified Gullstrand eyes) were created in an experimental study, using averaged ocular refractive data from a major epidemiologic study known as the Reykjavik Eye Study (Olsen et al., Acta Ophthalmol Scand, 85(4), 361-366, 2007) to calculate throughput and image magnification, as well as the respective retinal illuminance of each eye.

In this particular example, each model eye was created, with corrective lens correction, cornea and an axial length, such as to calculate retinal illuminance (RI) in two typical eyes of different refractive errors—one hyperopic and the other myopic. However, the calculation process can be applied to an eye of any refractive error for determining the respective RI.

In a specific, non-limiting example, the first eye was assigned an axial length (x) of 22 mm and, as per the Reykjavik Eye Study would most likely be associated with a corneal power (K) of 44 D and a lens power (L) of 24 D. The second eye was assigned an axial length of 27 mm and, similarly, corneal and lens powers of 41 D and 17 D, respectively. The associated corrective lens correction for these two eyes (based on averaged data from the Reykjavik eye study) would be +3 D and −3 D, respectively.

Note that different values may be used for the various optical parameters of the model eyes, without departing from the scope of the present invention. Also note that, although each model eye may also be assigned a crystalline lens power, ultimately that particular power does not influence the calculations in such a simple model eye (see below).

Continuing with the specific, non-limiting example, for a given vertex distance (14 mm), anterior chamber depth (3 mm) and pupil diameter (3 mm), basic geometric optics dictate that throughflux in a converging lens system (+3 D corrective lens and 44 D cornea) will be greater than the throughflux in a diverging lens system (−3 D corrective lens and 41 D cornea).

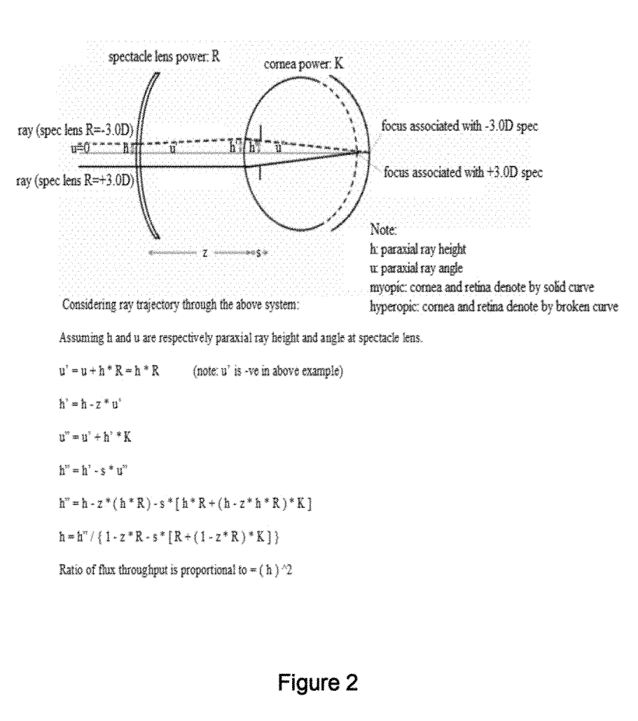

FIG. 2 is a schematic illustration of throughflux in a simplified Gullstrand eye, where the cornea and retina are denoted by a solid curve for the myopic model and by a broken curve for the hyperopic model.

Specific to the present invention, the following equations may be used to compute the throughflux for each eye, considering the ray trajectory through the optical system:

u′=u+h*R=h*R (negative in example of FIG. 2) (1)

h′=h−z*u′ (2)

u″=u′+h′*K/n (3)

h″=h′−s*u″ (4)

h″=h−z*(h*R)−s*[h*R+(h−z*h*R)*K/n] (5)

h=h″/{1−z*R−s*[R+(1−z*R)*K]} (6)

where:

h: paraxial ray height

u: paraxial ray angle (at corrective lens)

R: corrective lens power

K: corneal power

z: vertex distance

s: anterior chamber distance

n: index of refraction of the cornea anterior chamber complex

Since the ratio of the throughflux (T) is proportional to h2, the throughflux ratio for the hyperopic eye model (characterized by R=+3.0 D, K=44 D, z=14 mm, s=3 mm, h″=1.5 mm) and the myopic eye model (characterized by R=−3.0 D, K=41 D, z=14 mm, s=3 mm, h″=1.5 mm) can be calculated and, in this specific example, it is determined that the myopic eye has a throughflux 0.8 times that of the hyperopic eye.

With regard to the image magnification for each eye, once the rays are in the posterior chamber (see FIG. 1), their distribution on the retina (rays or photons/mm²) will determine the image magnification. The image size difference between the two model eyes is the result of the magnification difference, caused firstly by the corrective lens difference (spectacle magnification), and secondly by the optics of the eye itself (ocular magnification). In the case of the above non-limiting example of the hyperopic and myopic eye models, using simple geometric optical equations, U+P=V where U is object vergence at the lens, P is lens power and V is image ray vergence at the lens and magnification=UN, the +3 D lens at a vertex distance of 14 mm will result in spectacle magnification of 1.04 and the −3 D lens a magnification of 0.96 (in both cases, by sign convention, U and V are negative. The ratio 0.96/1.04 means the −3 D lens will result in an image that is 0.92× the size of the image from the +3 D lens. See Rubin, Optics for Clinicians, Triad publishing Co., 1974.

The axial length difference causes the retinal image to be:

27/22=1.23× larger in the myopic eye

where image magnification caused by the eye is a direct function of the axial length (per Bengtsson et al., Graefes Arch Clin Exp Ophthalmol, 230(1), 24-28, 1992; and Garway-Heath et al., Br J Ophthalmol, 82(6), 643-649, 1998).

The ratio of the linear retinal image sizes, and thus of the image magnifications, is the product of these magnifications, notably 1.13×.

Accordingly, we can calculate the RI for an eye by applying the determined T and IM values for that eye to the equation T/IM². In the case of this specific, non-limiting example of the hyperopic and myopic eye models, we can calculate the IR ratio (ratio of T/IM²) for the two eyes to be 0.8/(1.13)²× or 0.8/1.19×, making the retinal illumination in our myopic eye 0.63× that of the non-myopic eye. In other words, the myopic eye (−6 D difference from the hyperopic eye) receives just 63% of the retinal illuminance of the hyperopic eye.

In a variant, non-limiting example of implementation of the present invention, an optical system design/analysis software (e.g. OPTICSOFT-II, PhacoOptics®) may be used to compute the retinal illuminance of an eye. Using the ray-tracing feature of such an optical design software, it is possible to input the optical parameters of an eye and to calculate a true refractive error in the eye, as well as the retinal illuminance (or photonic intensity) of the eye.

Continuing with the above specific example of using averaged biometric data from the Reykjavic Eye Study, the same optical values as those discussed above with regard to the simplified Gullstrand models (e.g. corneal power, lens power, axial length, corrective lens power), as well as optionally additional parameters (e.g. lens thickness, refractive indices of ocular tissues, etc.) can be input to an optical design software, such as OPTICSOFT-II, which is capable to derive curvatures (e.g. anterior and posterior curvatures) for the lens of the eye while respecting an accepted predefined ratio of refractive power for these surfaces of the eye (e.g. anterior surface has 35% of the refractive power, while the posterior surface has 65% of the refractive power).

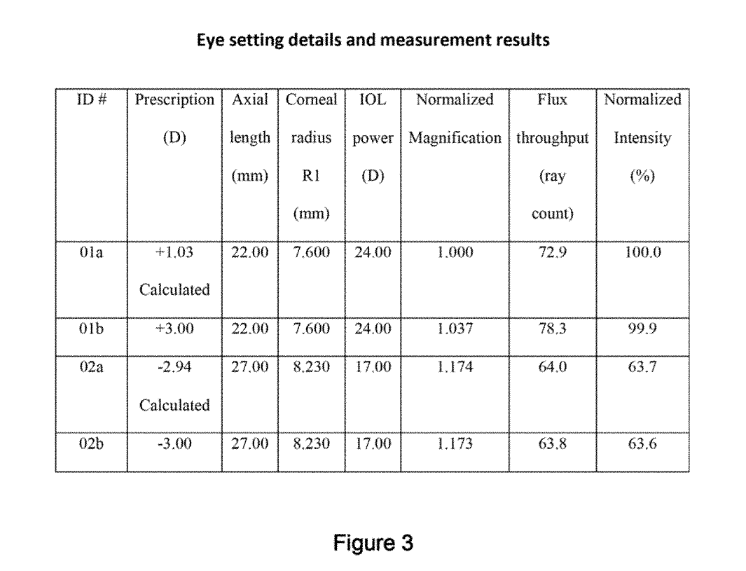

FIG. 3 illustrates the eye settings input to the OPTICSOFT-II ray-tracing software program in accordance with the above specific example of myopic and hyperopic eye models, as well as the measurement results output by the optical design software. The computer model gives a throughflux (ray count) measurement for each different eye setting, and shows the throughflux in the most myopic eye to be 82% that of the most hyperopic eye (note that this value was 80% using the above-described simpler geometric optic model). The computer model shows the RI in the most myopic eye to be 63.6% that of the most hyperopic eye (note that this ratio was 63% in the simpler geometric optic model). These results confirm the significant increase in retinal illuminance between a typical hyperopic eye and a typical myopic one, using averaged refractive parameters.

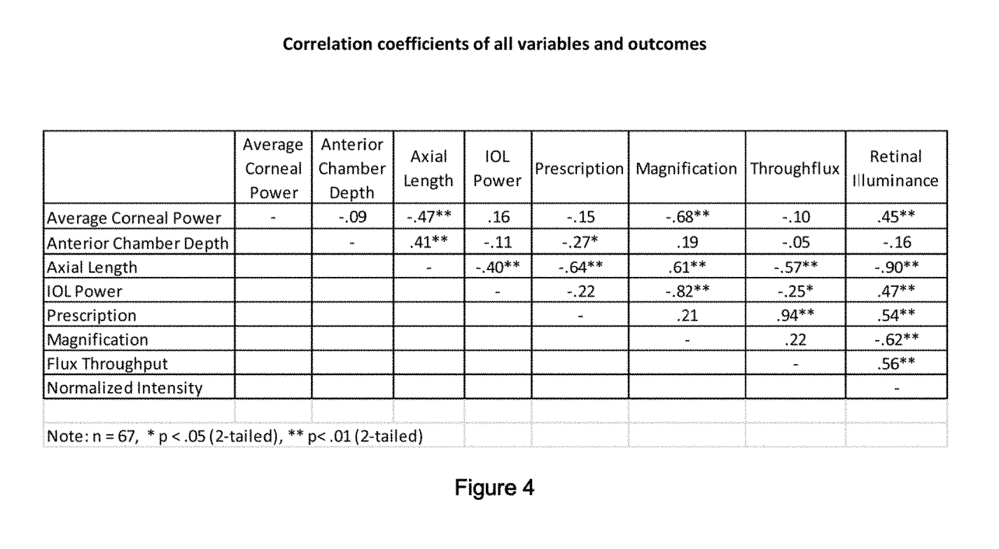

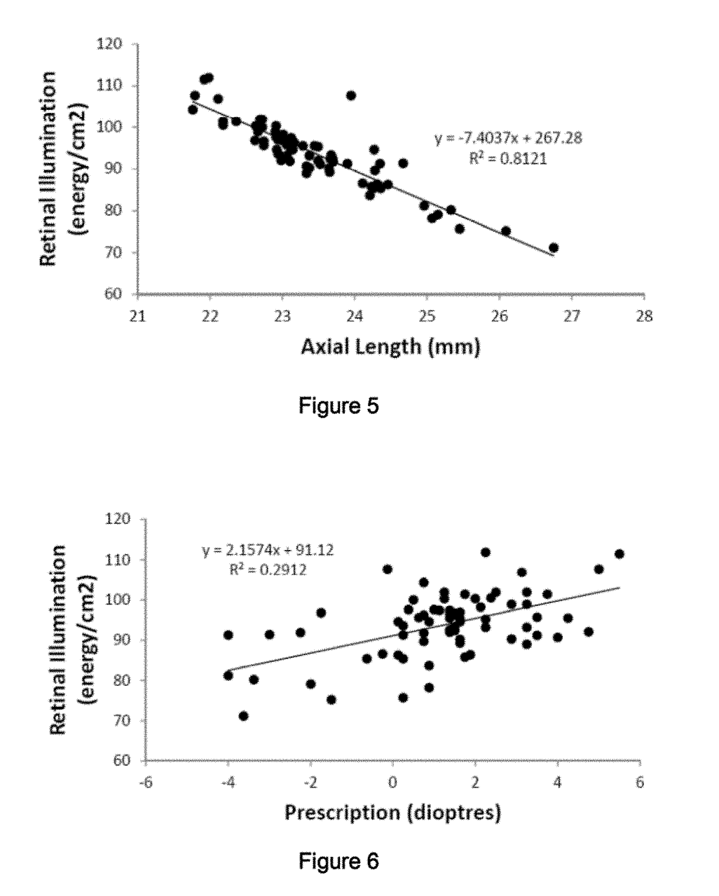

Accordingly, the results from a simple Gullstrand eye model, as can be confirmed using a ray-tracing software program, indicate that retinal illumination is markedly increased in a typical hyperopic eye compared to a typical myopic one. A more detailed examination of the eye's refractive variables using a random sample from the population-based Reykjavik eye study reveals that this increase in retinal illumination is highly inversely correlated with the axial length of the eye, as well as its corrective lens refraction, as illustrated by the results shown in FIGS. 4, 5 and 6. In one non-limiting example, the RI is 7.1% less for every mm increase in axial length and 2.2% greater for every diopter increase of corrective lens refraction.

In light of the foregoing, it can be concluded that the retinal illuminance level (or photon intensity level) of an eye varies from person to person, on a basis of the refractive parameters of each person and is especially correlated (inversely) to the axial length of an individual's eye. Since it is reasonable to conclude from the above that an individual's retinal illuminance level may significantly affect his/her chances of incurring retinal damage over time, it is proposed that this relative retinal illuminance level be considered when prescribing and/or manufacturing eyewear to protect the individual's ocular health. More specifically, a level of ocular protection required to reduce the light intensity of an eye should be determined on a basis of the retinal illuminance of the individual.

Specific to the present invention, the retinal illuminance level for an individual is used to prescribed a light transmission characteristic of eyewear lenses for the individual, such as to decrease the light intensity in the individual's eyes.

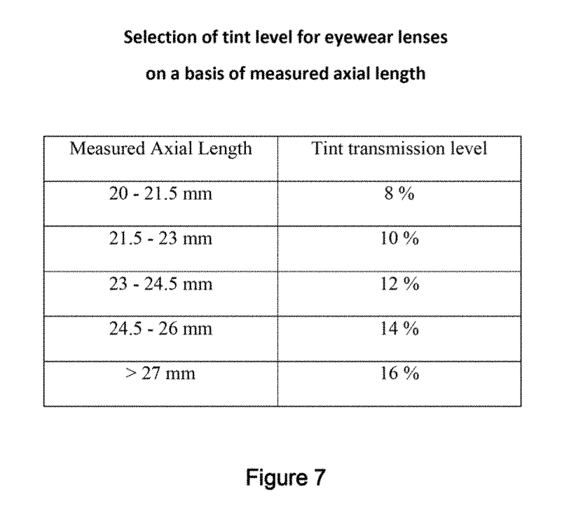

In a specific, non-limiting example of implementation, the light transmission characteristic of the lenses that is prescribed on a basis of the measured or calculated retinal illuminance level, and thus on the basis of at least one refractive parameter of the individual's eyes, is a tint level, which defines a specific level of tint for the lenses that is required to decrease the photonic intensity in the individual's eyes. In one example, a minimum tint level for eyewear lenses for the individual is selected on a basis of at least one refractive parameter of the individual's eyes. For example, as shown in FIG. 7, the axial length of the individual's eyes can be measured and a tint level can be selected as a function of of that axial length. Retinal illuminance inversely correlates to axial length. Thus light transmission characteristics of glasses (e.g. sunglasses, since sunlight is the major source of high energy radiance falling on the retina) can be recommended depending on one's axial length or spectacle refraction (the latter being a simplified substitute measurement for axial length). Eyes can be grouped by axial length 20-21.5 mm, 21.5-23, 23-24.5, 24.5-26 and 27 and higher. The shortest axial lengths would have transmission of 8%, next 10% and so on.

In another broad embodiment of the invention, there is provided a novel method for manufacturing eyewear for an individual, based on the individual's eyes relative retinal illuminance level. Basically, once an individual's retinal illuminance level has been determined, and the corresponding RPF value or minimum recommended tint level for that individual identified, it is possible to manufacture protecting eyewear for the individual that will reduce the individual's chances of incurring retinal damage due to overexposure to UV radiation and/or HEV light.

As is well known in the art, once an individual's eyes have been examined and an optical prescription for optimal vision has been generated, lenses are produced on a basis of this prescription, to fit an eyewear frame selected by the individual. The manufacturing process for the lenses includes grinding of optical curves into the back of the lenses on a basis of the optical prescription and polishing of the lenses, as well as beveling of the lenses to fit the latter to the selected eyewear frame, and tinting or treating the lenses as required before inserting them into the frame. Since these standard manufacturing steps for eyewear lenses are well known to those skilled in the art, they will not be described in further detail herein.

Specific to the present invention, once the lenses have been shaped (ground, polished and beveled), they undergo a tinting process to apply the minimum recommended tint level thereto. More specifically, the optical prescription resulting from an examination of the individual's eyes includes an MPF value for the individual, and thus the corresponding minimum tint level that the individual should be wearing on a regular basis in order to minimize retinal illuminance and hence protect his/her eyes from developing AMD. Though a minimum recommended tint level is identified for the individual on a basis of his or her relative retinal illuminance, it is the individual who ultimately selects a tint for the lenses of his or her eyewear.

Note that an individual may choose to have lenses generated with a tint level that is darker than the minimum recommended tint level. This would of course still minimize risk for developing retinal damage. However, choosing to tint the lenses with a tint level that is lighter than the minimum recommenced tint level would put the individual at greater risk of developing retinal damage, given that individual's risk based on his axial length (or other refractive parameter(s) used to calculate retinal illuminance), but may be what is required by the individual to properly function in everyday life.

The tinting process includes dipping the lenses into a container (e.g. heated metal bin) of the desired tint, for coating the lenses with the tint. Once dry, the eyeglass lenses are ready for insertion into the desired frame, after which the eyewear is ready to be worn by the individual.

It is important to note that the above-described embodiments and examples of implementation of the present invention have been presented for illustration purposes but that additional variants and modification are possible and should not be excluded from the scope of the present invention.

What is claimed is:

1. A method of prescribing eyewear for an individual, said method comprising:
a. determining a relative retinal illuminance level for the individual;
b. selecting, on a basis of the determined retinal illuminance level, a light transmission characteristic for eyewear lenses for the individual.

2. A method of prescribing eyewear as defined in claim 1, wherein said light transmission characteristic is a light reduction level.

3. A method of prescribing eyewear as defined in claim 1, wherein said light transmission characteristic is a minimum tint level.

4. A method of prescribing eyewear as defined in claim 3, further comprising using said determined retinal illuminance level to assign a retinal protection factor value to the individual, said retinal protection factor value indicative of the minimum tint level selected for the individual.

5. A method of prescribing eyewear as defined in claim 1, wherein determining a retinal illuminance level for the individual includes measuring at least one refractive parameter of the individual's eye.

6. A method of prescribing eyewear for an individual, said method comprising:
a. measuring at least one refractive parameter of the individual's eye;
b. determining, on a basis of the measured at least one refractive parameter, a minimum tint level for eyewear lenses for the individual.

7. A method as defined in claim 6, wherein said measuring at least one refractive parameter includes measuring at least one of a corrective lens power, a corneal power, a lens power, an anterior chamber depth and an axial length.

8. A computer-implemented method of calculating a light transmission characteristic for eyewear lenses for an individual comprising:
a. inputting measured refractive parameters of the eyes of the individual;
b. calculating the individual's retinal illuminance level based on a model of the eye; and
c. identifying a minimum light transmission characteristic for the eyewear lenses.

9. A method of manufacturing eyewear lenses for an individual, the individual's eyes having a specific retinal illuminance level, said method comprising:
a. using the individual's retinal illuminance level to identify a minimum tint level for the lenses;
b. producing a pair of lenses to fit an eyewear frame selected by the individual;
c. applying at least said minimum light transmission characteristic to the lenses.

10. A method as defined in claim 9, wherein said producing a pair of lenses includes grinding optical curves into the lenses on a basis of an optical prescription for the individual.

11. A method as defined in claim 9, wherein said applying at least said minimum light transmission characteristic to the lenses is done by applying a tinting process.

12. A method as defined in claim 10, wherein said applying at least said minimum light transmission characteristic to the lenses is done by applying a tinting process.
